(19) United States
(12) Patent Application Publication
(10) Pub. No.: US 2016/0310434 A1
(43) Pub. Date: Oct. 27, 2016

(54) GASTRO-RETENTIVE ORAL PHARMACEUTICAL COMPOSITIONS

(71) Applicant: ETHYPHARM, SAINT-CLOUD (FR)

(72) Inventors: CATHERINE HERRY, SAINT-OUEN DU TILLEUL (FR); PAULINE CONTAMIN, LA FEUILLIE (FR)

(73) Assignee: ETHYPHARM, SAINT-CLOUD (FR)

(21) Appl. No.: 15/104,698

(22) PCT Filed: Dec. 18, 2014
(86) PCT No.: PCT/EP2014/078597

§ 371 Date: Jun. 15, 2016

(30) Foreign Application Priority Data
Dec. 18, 2013 (FR) 1362916

(51) Int. Cl. A61K 9/20; A61K 9/00; A61K 31/197

(52) U.S. Cl. CPC A61K 9/2077; A61K 31/197; A61K 9/0065; A61K 9/2054; A61K 9/205; A61K 9/2018; A61K 9/2009

(57) ABSTRACT

The invention relates to a pharmaceutical matrix tablet which can be administered orally once or twice per day with gastro-retentive controlled release of Baclofen.

### CROSS-REFERENCE TO RELATED APPLICATIONS

This application is the U.S. National stage filing of International Application No. PCT/EP2014/078597, filed 18 Dec. 2014, and claims priority of French application number 1362916, filed 18 Dec. 2013, the entireties of which applications are incorporated herein by reference.

### FIELD OF THE INVENTION

The invention relates to an oral galenic formulation of a BCS class III molecule like baclofen in the form of a gastroretentive controlled-release tablet. These compositions can be administered once or twice per day.

### BACKGROUND OF THE INVENTION

The oral route is the first envisaged route when developing a novel pharmaceutical entity since it is conducive to promoting treatment compliance. However, this route is being abandoned for many molecules under development due to their low oral bioavailability. This may be due to various factors related to the very properties of the molecule or to the physiology of the gastrointestinal tract (Fasinu, et al., 2011). Various approaches, including physical or chemical means, have been investigated over the last ten years to improve the oral bioavailability of these molecules.

The Biopharmaceutics Classification System (BCS) is a significant tool used for the development of oral forms in the pharmaceutical industry and has been adopted in particular by the FDA, EMEA and WHO (Dahan, et al., 2009). It is divided into four categories of molecules and is based on the fundamental elements that control oral absorption, i.e., intestinal membrane permeability and the solubility of a molecule in the gastrointestinal medium. A molecule is considered soluble if the maximum dose of an immediate—release form is soluble in 250 ml or less of an aqueous medium the pH of which is between 1.2 and 6.8, and is considered permeable if its absorption through the intestinal membrane is greater than or equal to 90%. A BCS class III molecule has high solubility and low permeability. The latter characteristic is the factor limiting oral bioavailability and may lead to abandoning the development of an oral formulation of molecules nevertheless having strong therapeutic potential.

Baclofen (below) is a BCS class III molecule. Its physicochemical characteristics are summarized in Table 1.

TABLE I
Physicochemical characteristics of baclofen (internal report)

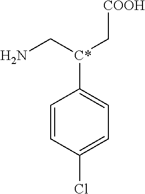

| Molar mass | 213.66 g/mol |
|---|---|
| Melting point | 180-191° C. |
| pH of a saturated solution | 6.5 |
| pKa | pKa₁ = 3.87 |
| | pKa₂ = 9.62 |
| Solubilities | Soluble: 0.1N HCl, 0.1N NaOH |
| | Slightly soluble: water (3 mg/ml) |
| | Very slightly soluble: ethanol, methanol |
| Log P | −1 |

At usual therapeutic doses, baclofen is known to have good oral bioavailability. However, its low log P indicates some hydrophilicity and thus low permeability. This observation is explained by the presence of specific transporters at the small intestine allowing the molecule to pass. This is even greater at the jejunum. Also, weak passage at the colon suggests the presence of another transport mechanism through the intestinal barrier (Merino, et al., 1989). This region of the digestive system contains no transporters but since the molecule is hydrophilic and of small size, weak passive transport through the tight junctions is possible. It thus appears that when a higher plasma concentration of baclofen is required, the taking of a higher dose is not effective due to saturation of the transporters. The clinical solution is thus to take small doses at shorter time intervals, which can quickly become burdensome for the patient.

An increase in the lipophilicity of baclofen, and thereby its transcellular permeability, was observed during the production of baclofen ester prodrugs (Leisen, et al., 2003). Through these properties, a higher concentration of prodrug was found at the target tissue—the brain—due to easier crossing of the blood—brain barrier. However, greater affinity for the efflux pump P—gp is to be noted as well as partial hydrolysis of the prodrug to baclofen which finally leads to a lower level of baclofen at the site of action after administration of the prodrug compared with administration of baclofen alone.

A baclofen absorption window was shown at the small intestine due to the presence of transporters (Merino, et al., 1989). To increase bioavailability, this absorption window could thus be used to advantage for galenic techniques allowing the oral form to be retained at or upstream of the window (Davis, 2005). Indeed, a longer residence time at the absorption site should in theory allow a greater passage of molecules across the intestinal barrier if they are not likely to undergo presystemic degradation before being absorbed. Thus, bioadhesive forms adhering to the intestinal mucus through the use of cationic polymers such as chitosan, or indeed gastroretentive forms which swell and float in the stomach, have emerged in recent years in pharmaceutical development.

Conventional prolonged—release dosage forms are thus hardly suitable.

The principle of gastroretentive forms consists in trapping the formulation in the stomach for a prolonged period and slowly releasing the active ingredient in the stomach, i.e., immediately upstream of the absorption window, which allows a prolonged absorption of the active substance.

To that end, gastroretentive forms must float in the alimentary bolus contained in the stomach in order not to undergo gastric emptying and to swell sufficiently not to pass the pylorus.

Generally, gastric emptying time in the fasting state occurs over a period of 0 to 2 hours and after having eaten over a period of 4 to 6 hours.

Gastroretentive formulations are known in the prior art.

U.S. Pat. No. 6,797,283 discloses the use of multilayer tablets consisting of a high-swelling layer and a layer containing the active ingredient. The tablet is thus retained in the stomach for a prolonged period.

U.S. Pat. No. 6,635,280 discloses tablets sufficiently small to be ingested which swell after ingestion.

EP2262484 describes pharmaceutical compositions in the form of gastroretentive tablets with prolonged release of two active ingredients, an opioid combined with acetaminophen. These tablets are monolithic with slow release and break down by erosion or they combine a fast—release part with another slow—release part in the form of a double—layer tablet. The retentive action is provided by a polymer matrix consisting of at least a hydrophilic polymer which swells when saturated with liquid to a size sufficient for gastric retention. However, these tablets can be evacuated by gastric emptying even before having swelled sufficiently not to pass the pylorus. For this reason, they must be administered with a meal.

EP 1745775 describes pharmaceutical compositions in the form of gastroretentive tablets comprising a granulated active substance with a mixture of a weak gelling agent, a strong gelling agent and a gas—generating agent. However, it was observed that these tablets can also be evacuated by gastric emptying because it takes too long for the tablets to float.

### SUMMARY OF THE INVENTION

The object of the invention is to provide controlled—release matrix—type baclofen tablets which remain in the stomach for at least 2 hours, preferably 4 hours, because they float, and this in less than several minutes.

### DETAILED DESCRIPTION OF THE INVENTION

Surprisingly, the inventors discovered that a gas generator is essential to the formulation of such a matrix tablet. However, it was observed that the more the amount of gas generator decreases to 5% by weight of the total weight of the tablet, the more the floating properties (floating time) are improved.

Moreover, the inventors discovered that a superdisintegrant is essential to the formulation of such a matrix tablet.

In addition, the inventors discovered that the presence of 10% by weight of the total weight of the tablet of sodium alginate is unfavorable to floating.

The present invention has as an object a pharmaceutical composition her the form of a gastroretentive tablet with improved floating, containing baclofen, usable for administration once or twice per day depending on the matrix used. These compositions overcome the disadvantages mentioned above.

The invention is characterized by the fact that in contact with gastric fluid, the composition quickly floats. This makes it possible to ensure a longer residence time of the tablet in the stomach and to ensure that the greatest proportion of active ingredient contained in the pharmaceutical composition is released and absorbed in the upper portion of the tract.

The present invention has as an object a matrix—type pharmaceutical tablet administrable by oral route one or two times per day with controlled release of baclofen by gastric retention comprising granules of baclofen and one or more diluents within a matrix comprising at least a gelling agent, a gas—generating agent and a superdisintegrant.

The gas—generating agent generates gas bubbles confined in the superdisintegrant which then swells in contact with gastric fluid up to twice its initial volume. The gelling agents generate a network of prolonged release of the active ingredient.

The formulation of the invention is particularly advantageous due the extremely short time it takes to start floating. Indeed, if the formulation takes too long to start floating, it is drained. The formulation of the invention has improved floating.

By time to start floating is meant the amount of time after which the tablets rise to the surface of the medium (timed by visual observation) during the type II dissolution test with paddle stirring (at a rotational speed of 100 rpm) as described in the European Pharmacopoeia 7th Edition (monograph 2.9.3). The tablets then remain on the surface throughout the dissolution test.

By improved floating is meant a time to start floating of less than 30 minutes in fed—mode gastric medium (pH between 3 and 5) and of less than 10 minutes in fasting—mode gastric medium (pH between 1 and 2).

The term “prolonged release” in the present application is used to indicate a release profile of the active ingredient which is modified relative to that which the active ingredient would have had alone in an immediate—release system.

Preferably, the prolonged release according to the invention is between 4 hours and 24 hours depending on the composition of the polymer matrix.

By “matrix-type tablet” is meant a pharmaceutical composition for oral administration containing an active substance dispersed uniformly in one or more excipients which, after compression, allow the formation of a matrix able to slow the release of the active ingredient.

The diluents suitable for baclofen granulation are cellulose derivatives such as microcrystalline cellulose like Avicel PH102, polyols like mannitol, calcium carbonate. Preferably, baclofen is granulated with mannitol.

By “gelling agent” or “agent controlling release” is meant vegetable gums of glucidic nature, linear polysaccharides (extracts of algae), substituted linear polysaccharides (galactomannans, xanthan gum), polyethylene glycol (polyethylene oxides), hydrophilic cellulose esters such as methylcellulose, hydroxypropylcellulose and hydroxypropylmethylcellulose. The function of this gelling agent in the present invention is to form a network able to retain the formation of gas that is generated when the tablet contacts the gastric medium and to ensure controlled release. The tablet according to the invention can also comprise a mixture of gelling agents.

The tablet according to the invention comprises from 5 to 50% by weight of gelling agent relative to the total weight of the tablet, preferably from 8 to 40%, particularly preferably from 10 to 30% by weight relative to the total weight of the tablet.

According to the invention the preferred gelling agents are xanthan gum, polyethylene oxides (PEO), of a molecular weight of about 4,000,000 g/mol (Polyox™ WSR301), of a molecular weight of about 900,000 g/mol (Polyox™ WSR1105), of a molecular weight of about 700,000 g/mol (Polyox™ WSR303), or cellulose ethers such as Methocel™ which are hydrosoluble polymers consisting of a mixture of methylcellulose and hydroxypropylmethylcellulose like Methocel™ K4M, Methocel™ K15M, Methocel™ K100M.

Preferably, the tablet according to the invention comprises less than 5% by weight of the total weight of the tablet of sodium alginate.

Particularly preferably, the tablet according to the invention does not comprise sodium alginate.

By “gas—generating agent” according to the invention is meant a compound that generates gas when it is in contact with an acidic medium such as gastric juice. The gas—generating agent according to the invention is selected from the group comprising carbonates such as sodium carbonate and sodium glycine carbonate, bicarbonates such as sodium bicarbonate (NaHCO₃), potassium bicarbonate, sulfites such as sodium sulfite, sodium bisulfite, sodium metabisulfite, and mixtures thereof.

Preferably, the gas—generating agent is sodium bicarbonate.

The tablet according to the invention comprises from 2 to 25% by weight of gas generator relative to the total weight of the tablet, preferably from 3 to 15%, from 4 to 10%, particularly preferably 5% by weight relative to the total weight of the tablet.

By superdisintegrant is meant polymers able to capture the surrounding liquid quickly; they comprise for example crosslinked cellulose polymers, crosslinked carboxymethylcellulose or croscarmellose, crospovidone or crosslinked polyvinylpolypyrrolidone which is a crosslinked N—vinyl-2—pyrrolidinone homopolymer. For example, mention may be made of crospovidone marketed under the name Kollidon®CL or also under the name Polyplasdone™.

The tablet according to the invention comprises from 1 to 30% by weight of superdisintegrant relative to the total weight of the tablet, preferably from 1 to 20%, from 2 to 15%, particularly preferably from 3 to 10% by weight relative to the total weight of the tablet.

According to the invention, the preferred superdisintegrant is croscarmellose, crospovidone or crosslinked polyvinylpolypyrrolidone which is a crosslinked N—vinyl-2—pyrrolidinone homopolymer, crospovidone marketed under the name Kollidon®CL or also under the name Polyplasdone™.

According to a preferred embodiment of the invention, the tablet according to the invention comprises the gelling agent and the gas generator in a weight ratio of gelling agent to gas generator of 1:10 to 10:1.

Particularly preferably, the tablet according to the invention comprises a superdisintegrant which is croscarmellose or crospovidone in proportions of between 3% and 8% by weight of the total weight of the tablet, a gas—generating agent which is sodium bicarbonate in proportions of between 4% and 8% by weight of the total weight of the tablet, a gelling agent which is either xanthan gum, Polyox™ or Methocel™ in proportions of between 10% and 30% by weight of the total weight of the tablet.

In general, the matrix of the tablet of the invention also comprises one or more diluents selected from cellulose derivatives such as microcrystalline cellulose, polyols like mannitol, calcium carbonate.

The matrix of the tablet of the invention can thus comprise from 5 to 75%, preferably from 10 to 60%, particularly preferably from 15 to 50% by weight relative to the total weight of the tablet of one or more diluents.

The matrix of the tablet of the invention can thus comprise any excipient known to modulate the controlled release of the active ingredient. Thus, the person skilled in the art will be able to adjust the composition so that it is adapted to administration once per day or twice per day.

The tablet of the invention can also comprise any excipient adequate and necessary to the manufacture of the tablet, such as lubricants like magnesium stearate or sodium stearyi fumarate and glidants such as talc or silica.

According to the invention, when the pharmaceutical compositions comprise a glidant and/or a lubricant, the glidant or the lubricant is present in proportions ranging from 0.2% to 2%, preferably in proportions ranging from 0.5 to 1.5% by weight of the total weight of the tablet.

The pharmaceutical composition according to the invention can comprise any additional conventional formulation excipient as well as flavors and dyes.

To the pharmaceutical composition according to the invention can also be applied a coating of polymer materials the aim of which is a simple protection against moisture or to provide coloring to differentiate dosages or a modulation of the release kinetics of the active ingredient from the polymer matrix according to techniques known to the person skilled in the art.

The tablets according to the invention are obtained by techniques well—known to the person skilled in the art.

In particular, granules are obtained by wet granulation by bringing baclofen together with one or more diluents and optionally in the presence of a binder. After drying, these granules are mixed uniformly with the excipients constituting the matrix and the whole is compressed.

The formulation according to the invention comprises from 10 mg to 300 mg of baclofen per dosage unit, preferably from 30 mg to 150 mg, able to be taken once or twice per day.

The invention also relates to a tablet according to the invention for use as a drug, preferably by oral route in a single dose once per day or in two doses per day.

Another object of the invention relates to a tablet according to the invention for use in the treatment of alcohol dependence or the maintaining of alcohol abstinence.

Another object of the invention relates to a method of treating alcoholism or of maintaining alcohol abstinence comprising the administration by oral route to an alcoholic or an abstaining patient once to twice per day of one or more, preferably one to two, tablets according to the invention.

### EXAMPLES

### 1. Example of a Composition According to Patent EP 1745775 (Comparative Excample)

In this example, the composition of the tablets respects the amounts described in patent EP 1745775.

TABLE 1
Centesimal composition similar to
that described in patent EP 1745775

| | | | % |
|---|---|---|---|
| Phase | Component | mg/unit | w/w |
| Wet | Baclofen | 30.0 | 6.0 |
| granulation | Xanthan gum (Vanzan^{R} NF) | 50.0 | 10.0 |
| | Microcrystalline cellulose and sodium | 50.0 | 10.0 |
| | carboxymethylcellulose (Avicel^{R} CL 611) | | |
| | Mannitol SD200 | 100.0 | 20.0 |
| | HPMC 603 (Pharmacoat^{R} 603) | 15.0 | 3.0 |
| Mixture | Mannitol SD 200 | 146.5 | 29.3 |
| | Sodium bicarbonate | 101.0 | 20.2 |
| | Precipitated silica (Syloid^{R} 244FP) | 2.5 | 0.5 |
| Lubrication | Magnesium stearate | 5.0 | 1.0 |
| | Total | 500.0 | 100.0 |

Baclofen is granulated with xanthan gum, Avicel CL611, mannitol SD200 and HPMC according to a wet granulation process in a high—shear mixer granulator with addition of a water/ethanol mixture in a ratio of 1:1% (w/w). After drying and sizing on an oscillating screen with a 425 μm mesh, the granulated active ingredient is mixed with the mixture excipients in a multidirectional mixer (Turbula 2 L) for 10 minutes then lubricated with magnesium stearate for 1 additional minute. The final mixture is then compressed on a rotary press (SVIAC PR12) equipped with round punches of 12 mm diameter and a forced—feed system.

The tablets are produced with a compression force of 18 kN and have a hardness of 105 N.

Table 2 presents the results of dissolution in 500 ml of pH 4.5 medium, according to USP test II (paddle stirring at 100 rpm with presence of a disk).

TABLE 2
Tablet dissolution test

| Time (h) | % dissolved | CV (%) |
|---|---|---|
| 1 | 43.00 | 12.26 |
| 2 | 69.25 | 4.65 |
| 3 | 75.11 | 3.97 |
| 4 | 80.08 | 2.28 |
| 6 | 87.87 | 2.42 |
| 12 | 100.88 | 0.88 |

During the dissolution test, the tablets were observed to float after 1 hour 45 minutes.

### 2. Example of a Composition with Sodium Alginate (Comparative Example)

In this example, sodium alginate was added to the composition.

TABLE 3
Centesimal composition of tablets comprising sodium alginate

| | | | % |
|---|---|---|---|
| Phase | Component | mg/unit | (w/w) |
| Wet | Baclofen | 30.0 | 6.0 |
| granulation | Mannitol SD200 | 120.0 | 24.0 |
| Mixture | Xanthan gum (Vanzan^{R} NF) | 37.5 | 7.5 |
| | Sodium alginate (Keltone^{R} LVCR) | 50.0 | 10.0 |
| | Crospovidone (Polyplasdone ™ | 37.5 | 7.5 |
| | Ultra) | | |
| | Sodium bicarbonate | 100.0 | 20.0 |
| | Microcrystalline cellulose PH102 | 65.0 | 13.0 |
| | (Avicel^{R} PH102) | | |
| | Calcium phosphate, dibasic dihydrate | 50.0 | 10.0 |
| | (Emcompress^{R}) | | |
| | Precipitated silica (Syloid^{R} 244FP) | 5.0 | 1.0 |
| Lubrication | Magnesium stearate | 5.0 | 1.0 |
| | Total | 500.0 | 100.0 |

Baclofen is granulated with mannitol SD200 according to a wet granulation process in a high—shear mixer granulator (Diosna P/VAC 10) with addition of purified water. After drying and sizing on an oscillating screen with a 425 μm mesh, the granulated active ingredient is mixed with the mixture excipients (except the lubricant) in a multidirectional mixer (Turbula 2 L) for 10 minutes, then lubricated with magnesium stearate for 1 additional minute. The final mixture is then compressed on a rotary press (Sviac PR12) equipped with round punches of 12 mm diameter and a forced—feed system. The tablets produced with a compression force of 20 kN have an average hardness of 103 N.

Table 4 presents the results of dissolution in 500 ml of pH 4.5 buffer medium, according to USP test II (paddle stirring at 100 rpm with presence of a disk).

TABLE 4
Dissolution test in 500 ml of pH 4.5 medium

| Time (h) | % dissolved | CV (%) |
|---|---|---|
| 1 | 25.35 | 9.58 |
| 2 | 44.45 | 2.02 |
| 4 | 57.37 | 1.20 |
| 8 | 70.91 | 2.56 |
| 10 | 76.35 | 0.83 |
| 16 | 89.82 | 0.71 |

During the dissolution test, the tablets were observed to float after 55 minutes to 75 minutes. Alginate at 10% w/w slows the floating time.

### 3. Example of Compositions without Sodium Bicarbonate (Comparative Example)

In this example, the mixture is prepared in the same manner as in Example 2. It is then compressed on a rotary press (Sviac PR12) equipped with round punches of 12 mm diameter and a forced—feed system. The centesimal composition is presented in Table 5.

TABLE 5
Centesimal composition of tablets without sodium bicarbonate

| | | | % |
|---|---|---|---|
| Phase | Component | mg/unit | (w/w) |
| Wet | Baclofen | 30.0 | 6.0 |
| granulation | Mannitol SD200 | 120.0 | 24.0 |
| Mixture | Xanthan gum (Vanzan^{R} NF) | 125.0 | 25.0 |
| | Crospovidone (Polyplasdone | 100.0 | 20.0 |
| | Ultra ™) | | |
| | Sodium bicarbonate | — | — |
| | Microcrystalline cellulose PH102 | 100.0 | 20.0 |
| | (Avicel^{R} PH 102) | | |
| | Mannitol SD200 | 15.0 | 3.0 |
| | Precipitated silica (Syloid^{R} 244FP) | 5.0 | 1.0 |
| Lubrication | Magnesium stearate | 5.0 | 1.0 |
| | Total | 500.0 | 100.0 |

The tablets are produced with a compression force of 16 kN and have an average hardness of 147 N. Table 6 presents the results of dissolution in 500 ml of pH 4.5 buffer medium, according to USP test II (paddle stirring at 100 rpm with presence of a disk).

TABLE 6
Dissolution test in 500 ml of pH 4.5 medium

| Time (h) | % dissolved | CV (%) |
|---|---|---|
| 1 | 12.26 | 2.10 |
| 2 | 20.25 | 2.35 |
| 4 | 33.98 | 1.32 |
| 8 | 65.84 | 14.41 |
| 12 | 81.76 | 12.77 |
| 20 | 93.32 | 6.18 |

The tablets do not float at any time during the dissolution test. Floating is never achieved. The formulation of Example 3 thus does not solve the technical problem.

### 4. Example with 5% of Gelling Agent

In this example, the mixture is prepared in the same manner as in Example 2. It is then compressed on a rotary press (Sviac PR12) equipped with round punches of 12 mm diameter and a forced—feed system. The composition is presented in Table 7.

TABLE 7
Centesimal composition of a tablet containing 5% of gelling agent

| | | | % |
|---|---|---|---|
| Phase | Component | mg/unit | (w/w) |
| Wet | Baclofen | 30.0 | 6.0 |
| granulation | Mannitol SD200 | 120.0 | 24.0 |
| Mixture | Xanthan gum (Vanzan^{R} NF) | 25.0 | 5.0 |
| | Crospovidone (Polyplasdone ™ Ultra) | 75.0 | 15.0 |
| | Sodium bicarbonate | 117.5 | 23.5 |
| | Microcrystalline cellulose PH102 | 95.5 | 19.1 |
| | (Avicel^{R} PH102) | | |
| | Mannitol SD200 | 27.0 | 5.4 |
| | Precipitated silica (Syloid^{R} 244FP) | 5.0 | 1.0 |
| Lubrication | Magnesium stearate | 5.0 | 1.0 |
| | Total | 500.0 | 100.0 |

The tablets are produced with a compression force of 21 kN and have an average hardness of 125 N. The results of the dissolution test in pH 4.5 buffer according to USP test II (paddle stirring at 100 rpm with presence of a disk) are presented in Table 8.

TABLE 8
Dissolution test in 500 ml of pH 4.5 medium

| Time (h) | % dissolved | CV (%) |
|---|---|---|
| 0.5 | 74.58 | 1.65 |
| 1 | 81.08 | 1.90 |
| 2 | 86.79 | 1.14 |
| 3 | 87.86 | 0.25 |
| 6 | 88.10 | 0.43 |

During the dissolution test, the tablets were observed to float after 30 seconds.

### 5. Example with 50% of Gelling Agent

In this example, the mixture is prepared in the same manner as in Example 2. It is then compressed on a rotary press (Sviac PR12) equipped with oblong punches of dimensions 19×7.5 mm and a forced—feed system. The centesimal composition is presented in Table 9.

TABLE 9
Centesimal composition of tablets
comprising 50% of gelling agent.

| Phase | Component | mg/unit | % |
|---|---|---|---|
| Wet | Baclofen | 30.0 | 4.6 |
| granulation | Mannitol SD200 | 133.1 | 20.5 |
| Mixture | HPMC K100M (Methocel ™ K100M) | 325.0 | 50.0 |
| | Crospovidone (Polyplasdone ™ Ultra) | 65.0 | 10.0 |
| | Sodium bicarbonate | 32.5 | 5.0 |
| | Microcrystalline cellulose PH102 | 51.4 | 7.9 |
| | (Avicel^{R} PH102) | | |
| | Precipitated silica (Syloid^{R} 244FP) | 6.5 | 1.0 |
| Lubrication | Magnesium stearate | 6.5 | 1.0 |
| | Total | 650.0 | 100.0 |

The tablets are produced with a compression force of 10 kN and have an average hardness of 133 N. Table 10 presents the results of dissolution in 500 ml of pH 1.2 buffer medium, according to USP test II (paddle stirring at 100 rpm with presence of a disk).

TABLE 10
Dissolution test in 500 ml of pH 1.2 medium

| Time (h) | % dissolved | CV (%) |
|---|---|---|
| 1 | 18.17 | 1.78 |
| 2 | 26.95 | 3.90 |
| 4 | 40.93 | 7.57 |
| 8 | 61.63 | 10.65 |
| 12 | 76.31 | 11.02 |
| 24 | 97.45 | 5.18 |

During the dissolution test, the 6 tablets were observed to float after 4 to 8 minutes.

### 6. Example with 30% of Gelling Agent

In this example, the mixture is prepared in the same manner as in Example 2. It is then compressed on a rotary press (Sviac PR12) equipped with round punches of 12 mm diameter and a forced—feed system. The composition is presented in Table 11.

TABLE 11
Centesimal composition of the tablet
comprising 30% of gelling agent

| Phase | Component | mg/unit | % |
|---|---|---|---|
| Wet | Baclofen | 30.0 | 7.1 |
| granulation | Mannitol SD200 | 120.0 | 28.2 |
| Mixture | HPMC K15M (Methocel ™ K15M) | 127.5 | 30.0 |
| | Crospovidone (Polyplasdone ™ Ultra) | 63.7 | 15.0 |
| | Sodium bicarbonate | 21.3 | 5.0 |
| | Microcrystalline cellulose PH102 | 54.0 | 12.7 |
| | (Avicel^{R} PH102) | | |
| | Precipitated silica (Syloid^{R} 244FP) | 4.3 | 1.0 |
| Lubrication | Magnesium stearate | 4.3 | 1.0 |
| | Total | 425.0 | 100.0 |

The tablets are produced with a compression force of 16 kN and have an average hardness of 116 N. Table 12 presents the results of dissolution in 500 ml of pH 1.2 buffer medium, according to USP test II (paddle stirring at 100 rpm with presence of a disk).

TABLE 12
Dissolution test in 500 ml of pH 1.2 medium

| Time (h) | % dissolved | CV(%) |
|---|---|---|
| 1 | 25.87 | 1.86 |
| 2 | 37.69 | 1.47 |
| 4 | 54.70 | 1.69 |
| 8 | 76.72 | 1.72 |
| 16 | 94.41 | 1.37 |

During the dissolution test, the tablets were observed to float after 15 seconds.

### 7. Example with 20% of Gelling Agent

In this example, the mixture is prepared in the same manner as in Example 2. It is then compressed on a rotary press (Sviac PR12) equipped with round punches of 12 mm diameter and a forced—feed system. The composition is presented in Table 13.

TABLE 13
Centesimal composition of the tablet
comprising 20% of gelling agent

| Phase | Component | mg/unit | % |
|---|---|---|---|
| Wet | Baclofen | 30.0 | 7.1 |
| granulation | Mannitol SD200 | 120.0 | 28.2 |
| Mixture | Polyethylene oxide (Polyox WSR 303) | 85.0 | 20.0 |
| | Crospovidone (Polyplasdone ™ Ultra) | 63.7 | 15.0 |
| | Sodium bicarbonate | 21.3 | 5.0 |
| | Microcrystalline cellulose PH102 | 96.4 | 22.7 |
| | (Avicel^{R} PH102) | | |
| | Precipitated silica (Syloid^{R} 244FP) | 4.3 | 1.0 |
| Lubrication | Magnesium stearate | 4.3 | 1.0 |
| | Total | 425.0 | 100.0 |

The tablets are produced with a compression force of 16 kN and have an average hardness of 116 N. Table 14 presents the results of dissolution in 500 ml of pH 4.5 buffer medium, according to USP test II (paddle stirring at 100 rpm with presence of a disk).

TABLE 14
Dissolution test in 500 ml of pH 4.5 medium

| Time (h) | % dissolved | CV (%) |
|---|---|---|
| 1 | 17.77 | 6.83 |
| 2 | 24.94 | 5.25 |
| 4 | 37.44 | 3.06 |
| 8 | 60.02 | 2.40 |
| 16 | 84.12 | 1.45 |

During the dissolution test, the tablets were observed to float after 6 minutes.

### 8. Example with 10% of Gelling Agent

In this example, the mixture is prepared in the same manner as in Example 2. It is then compressed on a rotary press (Sviac PR12) equipped with round punches of 12 mm diameter and a forced—feed system. The composition is presented in Table 15.

TABLE 15
Centesimal composition of the tablet
comprising 10% of gelling agent

| Phase | Component | mg/unit | % |
|---|---|---|---|
| Wet | Baclofen | 30.0 | 7.1 |
| granulation | Mannitol SD200 | 120.0 | 28.2 |
| Mixture | HPMC K100M (Methocel ™ K100M) | 42.5 | 10.0 |
| | Crospovidone (Polyplasdone ™ Ultra) | 21.3 | 5.0 |
| | Sodium bicarbonate | 21.3 | 5.0 |
| | Microcrystalline cellulose PH102 | 160.3 | 37.7 |
| | (Avicel^{R} PH102) | | |
| | Calcium carbonate | 21.3 | 5.0 |
| | Precipitated silica (Syloid^{R} 244FP) | 4.3 | 1.0 |
| Lubrication | Magnesium stearate | 4.3 | 1.0 |
| | Total | 425.0 | 100.0 |

The tablets are produced with a compression force of 15 kN and have an average hardness of 122 N. Table 16 presents the results of dissolution in 500 ml of pH 4.5 buffer medium, according to USP test II (paddle stirring at 100 rpm with presence of a disk).

TABLE 16
Dissolution test in 500 ml of pH 4.5 medium

| Time (h) | % dissolved | CV (%) |
|---|---|---|
| 1 | 39.99 | 1.18 |
| 2 | 52.25 | 0.62 |
| 4 | 68.45 | 0.43 |
| 8 | 87.17 | 0.59 |
| 16 | 96.87 | 1.36 |

During the dissolution test, the tablets were observed to float after 20 seconds.

### 9. Example with Croscarmellose Sodium

In this example, the mixture is prepared in the same manner as in Example 2. It is then compressed on a rotary press (Sviac PR12) equipped with round punches of 12 mm diameter and a forced—feed system. The composition is presented in Table 17.

TABLE 17
Centesimal composition of the tablet comprising croscarmellose sodium

| Phase | Component | mg/unit | % |
|---|---|---|---|
| Wet | Baclofen | 30.0 | 6.0 |
| granulation | Mannitol SD200 | 120.0 | 24.0 |
| Mixture | Xanthan gum (Vanzan^{R} NF) | 40.0 | 8.0 |
| | Croscarmellose sodium (AcDiSol^{R}) | 60.0 | 12.0 |
| | Sodium bicarbonate | 117.5 | 23.5 |
| | Microcrystalline cellulose PH102 | 95.5 | 19.1 |
| | (Avicel^{R} PH 102) | | |
| | Mannitol SD200 | 27.0 | 5.4 |
| | Precipitated silica (Syloid^{R} 244FP) | 5.0 | 1.0 |
| Lubrication | Magnesium stearate | 5.0 | 1.0 |
| | Total | 500.0 | 100.0 |

The tablets are produced with a compression force of 22 kN and have an average hardness of 141 N. Table 18 presents the results of dissolution in 500 ml of pH 4.5 buffer medium, according to USP test II (paddle stirring at 100 rpm with presence of a disk).

TABLE 18
Dissolution test in 500 ml of pH 4.5 medium

| Time (h) | % dissolved | CV (%) |
|---|---|---|
| 1 | 43.74 | 4.82 |
| 2 | 52.30 | 3.38 |
| 4 | 63.92 | 2.47 |
| 6 | 71.97 | 1.40 |
| 12 | 90.76 | 0.34 |

During the dissolution test, the tablets were observed to float after 30 seconds.

### 10. Example with 20% of Crospovidone

In this example, the mixture is prepared in the same manner as in Example 2. It is then compressed on a rotary press (Sviac PR12) equipped with round punches of 12 mm diameter and a forced—feed system. The composition is presented in Table 19.

TABLE 19
Centesimal composition of the tablet comprising 20% of crospovidone

| Phase | Component | mg/unit | % |
|---|---|---|---|
| Wet | Baclofen | 30.0 | 6.0 |
| granulation | Mannitol SD200 | 120.0 | 24.0 |
| Mixture | Xanthan gum (Vanzan^{R} NF)) | 125.0 | 25.0 |
| | Crospovidone (Polyplasdone ™ Ultra) | 100.0 | 20.0 |
| | Sodium bicarbonate | 25.0 | 5.0 |
| | Microcrystalline cellulose PH102 | 75.0 | 15.0 |
| | (Avicel^{R} PH 102) | | |
| | Mannitol SD200 | 15.0 | 3.0 |
| | Precipitated silica (Syloid^{R} 244FP) | 5.0 | 1.0 |
| Lubrication | Magnesium stearate | 5.0 | 1.0 |
| | Total | 500.0 | 100.0 |

The tablets are produced with a compression force of 18 kN and have an average hardness of 121 N. Table 19 presents the results of dissolution in 500 ml of pH 4.5 buffer medium, according to USP test II (paddle stirring at 100 rpm with presence of a disk).

TABLE 20
Dissolution test in 500 ml of pH 4.5 medium

| Time (h) | % dissolved | CV (%) |
|---|---|---|
| 1 | 10.16 | 1.08 |
| 2 | 16.67 | 0.97 |
| 4 | 28.28 | 1.04 |
| 8 | 49.11 | 0.88 |
| 12 | 67.63 | 1.43 |
| 20 | 96.81 | 1.01 |

During the dissolution test, the tablets were observed to float after 9 minutes.

### 11. Example with 90 Mg of Active Ingredient

In this example, the mixture is prepared in the same manner as in Example 2. It is then compressed on a rotary press (Sviac PR12) equipped with round punches of 12 mm diameter and a forced—feed system. The composition is presented in Table 21.

TABLE 21
Centesimal composition of the tablet comprising 90 mg
of active ingredient.

| Phase | Component | mg/unit | % |
|---|---|---|---|
| Wet | Baclofen | 90.0 | 18.0 |
| granulation | Mannitol SD200 | 60.0 | 12.0 |
| Mixture | HPMC K100M (Methocel ™ K100M) | 150.0 | 30.0 |
| | Crospovidone (Polyplasdone ™ Ultra) | 25.0 | 5.0 |
| | Sodium bicarbonate | 25.0 | 5.0 |
| | Microcrystalline cellulose PH102 | 90.0 | 18.0 |
| | (Avicel^{R} PH102) | | |
| | Calcium carbonate | 50.0 | 10.0 |
| | Precipitated silica (Syloid^{R} 244FP) | 5.0 | 1.0 |
| Lubrication | Magnesium stearate | 5.0 | 1.0 |
| | Total | 500.0 | 100.0 |

The tablets are produced with a compression force of 14 kN and have an average hardness of 115 N. Table 22 presents the results of dissolution in 500 ml of pH 1.2 buffer medium, according to USP test II (paddle stirring at 100 rpm with presence of a disk).

TABLE 22
Dissolution test in 500 ml of pH 1.2 medium

| Time (h) | % dissolved | CV (%) |
|---|---|---|
| 1 | 15.92 | 4.19 |
| 2 | 23.78 | 1.62 |
| 4 | 35.56 | 2.63 |
| 8 | 53.00 | 2.53 |
| 16 | 80.34 | 2.29 |
| 24 | 95.45 | 1.87 |

During the dissolution test in pH 1.2, the tablets were observed to float after 30 seconds.

### 12. Example with Colored Coating

In this example, the mixture is prepared in the same manner as in Example 2. It is then compressed on a rotary press (Sviac PR12) equipped with round punches of 12 mm diameter and a forced—feed system.

TABLE 23
Centesimal composition of the tablet comprising a colored coating

| Phase | Component | mg/unit | % |
|---|---|---|---|
| Wet | Baclofen | 30.0 | 5.8 |
| granulation | Mannitol SD200 | 120.0 | 23.3 |
| Mixture | HPMC K100M (Methocel ™ K100M) | 150.0 | 29.1 |
| | Crospovidone (Polyplasdone ™ Ultra) | 25.0 | 4.8 |
| | Sodium bicarbonate | 25.0 | 4.8 |
| | Microcrystalline cellulose PH102 | 90.0 | 17.5 |
| | (Avicel^{R} PH102) | | |
| | Calcium carbonate | 50.0 | 9.7 |
| | Precipitated silica (Syloid^{R} 244FP) | 5.0 | 1.0 |
| Lubrication | Magnesium stearate | 5.0 | 1.0 |
| Colored | Opadry II 85F230062 | 15.5 | 3.0 |
| coating | | | |
| | Total | 515.5 | 100.0 |

The tablets are produced with a compression force of 12 kN and have an average hardness of 88 N. The tablets are then coated in a perforated turbine (O'Hara Labcoat M) with 3% of Opadry II for coloring. After coating, the average hardness of the tablets is 140 N.

Table 24 presents the results of dissolution in 500 ml of pH 1.2 buffer medium, according to USP test II (paddle stirring at 100 rpm with presence of a disk).

TABLE 24
Dissolution test in 500 ml of pH 1.2 medium

| Time (h) | % dissolved | CV (%) |
|---|---|---|
| 1 | 15.94 | 1.74 |
| 2 | 24.90 | 1.06 |
| 4 | 37.88 | 1.18 |
| 8 | 56.40 | 0.55 |
| 16 | 85.78 | 0.74 |
| 24 | 97.10 | 1.54 |

The floating time found during the dissolution test in pH 1.2 is 4 minutes. The floating time found in the pH 4.5 buffer medium under the same stirring conditions is 18 minutes.

### 13. Example with 150 mg of Active Ingredient and a Pair of Gelling Agents

TABLE 25
Centesimal composition of the tablet comprising 150 mg of active
ingredient and a pair of gelling agents

| Phase | Component | mg/unit | % |
|---|---|---|---|
| Wet | Baclofen | 150.0 | 21.4 |
| granulation | Mannitol SD200 | 93.75 | 13.4 |
| | HPMC 603 (Pharmacoat 603) | 6.25 | 0.9 |
| Mixture | HPMC K100M (Methocel ™ K100M) | 70.0 | 10.0 |
| | HPMC K4M (Methocel ™ K4M) | 35.0 | 5.0 |
| | Crospovidone (Polyplasdone ™ Ultra) | 21.3 | 3.0 |
| | Sodium bicarbonate | 21.3 | 3.0 |
| | Microcrystalline cellulose PH200 | 267.1 | 38.2 |
| | (Avicel^{R} PH200) | | |
| | Calcium carbonate | 21.3 | 3.0 |
| | Precipitated silica (Syloid^{R} 244FP) | 7.0 | 1.0 |
| Lubrication | Magnesium stearate | 7.0 | 1.0 |
| | Total | 700.0 | 100.0 |

In this example, baclofen is granulated with mannitol SD 200 according to a wet granulation process in a high—shear mixer granulator (Diosna P/VAC 10) with addition of HPMC 603 and purified water. After drying and sizing on an oscillating screen with a 425 μm mesh, the granulated active ingredient is mixed with the excipients in the same way as in Example 2. It is then compressed on a rotary press (Sviac PR12) equipped with oblong punches of dimensions 19×7.5 mm and a forced—feed system.

The tablets produced with a compression force of 8 kN have an average hardness of 124 N. The table below presents the results of dissolution in 500 ml of pH 1.2 buffer medium, according to USP test II (paddle stirring at 100 rpm with presence of a disk).

TABLE 26
Dissolution test in 500 ml of pH 1.2 medium

| Time (h) | % dissolved | CV (%) |
|---|---|---|
| 1 | 30.03 | 7.52 |
| 2 | 44.13 | 3.64 |
| 4 | 62.24 | 3.23 |
| 8 | 84.40 | 2.82 |
| 16 | 100.45 | 2.86 |
| 24 | 101.28 | 1.51 |

The floating time found during the dissolution test in pH 1.2 is 5 seconds. The floating time found in the pH 4.5 buffer medium under the same stirring conditions is 20 seconds.

### 14. Example with 150 mg of Active Ingredient and a Pair of Gelling Agents

TABLE 27
Centesimal composition of the tablet comprising 150 mg of active
ingredient and a pair of gelling agents

| Phase | Component | mg/unit | % |
|---|---|---|---|
| Wet | Baclofen | 150.0 | 21.4 |
| granulation | Mannitol SD200 | 93.75 | 13.4 |
| | HPMC 603 (Pharmacoat 603) | 6.25 | 0.9 |
| Mixture | HPMC K100M (Methocel ™ K100M) | 70.0 | 10.0 |
| | HPMC K4M (Methocel ™ K4M) | 70.0 | 10.0 |
| | Crospovidone (Kollidon CL) | 35.0 | 5.0 |
| | Sodium bicarbonate | 35.0 | 5.0 |
| | Microcrystalline cellulose PH200 | 205.0 | 29.3 |
| | (Avicel^{R} PH200) | | |
| | Calcium carbonate | 21.0 | 3.0 |
| | Precipitated silica (Syloid^{R} 244FP) | 7.0 | 1.0 |
| Lubrication | Magnesium stearate | 7.0 | 1.0 |
| | Total | 700.0 | 100.0 |

In this example, baclofen is granulated with mannitol SD 200 according to a wet granulation process in a high—shear mixer granulator (Diosna P/VAC 10) with addition of HPMC 603 and purified water. After drying and sizing on an oscillating screen with a 425 μm mesh, the granulated active ingredient is mixed with the excipients in the same way as in Example 2. It is then compressed on a rotary press (Sviac PR12) equipped with oblong punches of dimensions 18×9 mm and a forced—feed system.

The tablets produced with a compression force of 21 kN have an average hardness of 202 N. The table below presents the results of dissolution in 500 ml of pH 1.2 buffer medium, according to USP test II (paddle stirring at 100 rpm with presence of a disk).

TABLE 28
Dissolution test in 500 ml of pH 1.2 medium

| Time (h) | % dissolved | CV (%) |
|---|---|---|
| 1 | 21.20 | 8.17 |
| 2 | 32.94 | 7.35 |
| 4 | 47.36 | 4.69 |
| 8 | 69.69 | 2.25 |
| 16 | 97.10 | 1.30 |
| 24 | 100.03 | 0.56 |

The floating time found during the dissolution test in pH 1.2 is 15 seconds. The floating time found in the pH 4.5 buffer medium under the same stirring conditions is 2 minutes.

### 15. Example with Colored Coating

TABLE 29
Centesimal composition of the tablet with colored coating

| Phase | Component | mg/unit | % |
|---|---|---|---|
| Wet | Baclofen | 30.00 | 7.76 |
| granulation | Mannitol SD200 | 43.13 | 11.16 |
| | HPMC 603 (Pharmacoat 603) | 1.88 | 0.49 |
| Mixture | HPMC K100M (Methocel ™ K100M) | 75.00 | 19.40 |
| | Crospovidone (Kollidon CL) | 28.13 | 7.28 |
| | Sodium bicarbonate | 28.13 | 7.28 |
| | Microcrystalline cellulose PH102 | 142.50 | 36.86 |
| | (Avicel^{R} PH102) | | |
| | Calcium carbonate | 18.75 | 4.85 |
| | Precipitated silica (Syloid^{R} 244FP) | 3.75 | 0.97 |
| Lubrication | Magnesium stearate | 3.75 | 0.97 |
| Colored | Opadry II 85F240070 | 11.25 | 3.00 |
| coating | | | |
| | Total | 386.3 | 100.0 |

In this example, baclofen is granulated with mannitol SD 200 according to a wet granulation process in a high—shear mixer granulator (Diosna P/VAC 10) with addition of HPMC 603 and purified water. After drying and sizing on an oscillating screen with a 425 μm mesh, the granulated active ingredient is mixed with the excipients in the same way as in Example 2. It is then compressed on a rotary press (Sviac PR12) equipped with oblong punches of dimensions 15×7 mm and a forced—feed system.

The tablets produced with a compression force of 10 kN have an average hardness of 135 N. The tablets are then coated in a perforated turbine (O′Hara Labcoat M) with 3% of Opadry II for coloring. After coating, the tablets have an average hardness of 166 N.

The table below presents the results of dissolution in 500 ml of pH 1.2 buffer medium, according to USP test II (paddle stirring at 100 rpm with presence of a disk).

TABLE 30
Dissolution test in 500 ml of pH 1.2 medium

| Time (h) | % dissolved | CV (%) |
|---|---|---|
| 1 | 26.3 | 6.02 |
| 2 | 39.3 | 3.03 |
| 4 | 54.3 | 3.09 |
| 8 | 81.4 | 3.90 |
| 16 | 99.6 | 0.88 |
| 24 | 100.0 | 0.97 |

The floating time found during the dissolution test in pH 1.2 is 10 seconds. The floating time found in the pH 4.5 buffer medium under the same stirring conditions is 40 seconds.

### BIBLIOGRAPHY

Dahan A, Miller J M and Amidon G L Prediction of solubility and permeability class membership: provisional BCS classification of the world's top oral drug [Review]//The AAPS Journal, vol. 11, no. 4.-2009.-pp. 740-746.
Davis S S Formulation strategies for absorption windows [Review]//Drug Discovery Today, vol. 10, no. 4.-2005.
Fasinu P [et al.] Diverse approaches for the enhancement of oral drug bioavailability [Review]//Biopharmaceutics and Drug Disposition, vol. 32.-2011.-pp. 185-209.
Merino M [et al.] Evidence of a specialized transport mechanism for the intestinal absorption of baclofen [Review]//Biopharmaceutics and Drug Disposition, vol. 10.-1989.-pp. 279-297.
Leisen CC [et al.] Lipophilicities of baclofen ester prodrugs correlate with affinities to the ATP-dependent efflux pump P-glycoprotein: relevance for their permeation across the blood-brain barrier? [Review]//Pharmaceutical Research, vol. 20, no. 5.-2003.-pp. 772-778.

1. A matrix-type pharmaceutical tablet administrable by oral route once to twice per day with controlled release of baclofen by gastric retention, comprising granules of baclofen and one or more diluents within a matrix comprising at least a gelling agent, a gas-generating agent and a superdisintegrant.

2. The tablet according to claim 1, comprising baclofen granules and mannitol within a matrix comprising at least a gelling agent, a gas-generating agent and a superdisintegrant.

3. The tablet according to claim 1, comprising from 1 to 30% by weight of superdisintegrant relative to the total weight of the tablet.

4. The tablet according to claim 1, comprising from 5 to 50% by weight of gelling agent relative to the total weight of the tablet.

5. The tablet according to claim 1, comprising from 2 to 25% by weight of gas generator relative to the total weight of the tablet.

6. The tablet according to claim 5, comprising 5% by weight of gas generator relative to the total weight of the tablet.

7. The tablet according to claim 1, not comprising sodium alginate.

8. The tablet according to claim 1, wherein the gelling agent and the gas generator are in a weight ratio of gelling agent to gas generator of 1:10 to 10:1.

9. The tablet according to claim 1, comprising:
granules comprising baclofen and mannitol within a
matrix comprising a superdisintegrant which is croscarmellose or crospovidone in proportions of between 3% and 8% by weight of the total weight of the tablet, a gas—generating agent which is sodium bicarbonate in proportions of between 4% and 8% by weight of the total weight of the tablet and a gelling agent which is either xanthan gum, Polyox™ or Methocel™ in proportions of between 10% and 30% by weight of the total weight of the tablet.

10. A method of treatment of alcohol dependence or of maintaining alcohol abstinence, comprising the administration once to twice per day of a tablet according to claim 1 to a patient in need thereof.

11. The tablet according to claim 2, comprising from 1 to 30% by weight of superdisintegrant relative to the total weight of the tablet.
